(19) United States
(12) Patent Application Publication
(10) Pub. No.: US 2004/0176733 A1
(43) Pub. Date: Sep. 9, 2004

(54) Method of mass-producing disposable absorbent articles exhibiting consistent absorbency characteristics and disposable absorbent articles made thereby

(75) Inventors: Frank S. Glaug, Chester Springs, PA (US); Robert T. Cole, Jackson, NJ (US)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(73) Assignee: Tyco Healthcare Retail Services AG

(21) Appl. No.: 10/383,431

(22) Filed: Mar. 7, 2003

(51) Int. Cl. A61F 13/15; A61F 13/20

(52) U.S. Cl. 604/378

(57) ABSTRACT

A method of making, e.g., mass-producing, disposable absorbent articles, e.g., diapers, exhibiting consistent fluid absorption characteristics and the articles produced thereby. The method comprises providing a top-sheet and a fluid absorbent core, e.g., pulp with SAP and a binder. A composite fluid acquisition system is provided between the top sheet and the core and comprises a first fluid acquisition layer, e.g., a three-dimensional apertured film, and a fluid absorbency enhancing layer of an air-laid substrate. The first fluid acquisition layer is located under the top-sheet and over the fluid absorbency enhancing layer at the fluid intake zone. A second fluid acquisition layer comprises a fibrous, fluid pervious material may be provided located under the first fluid acquisition layer at the fluid intake zone.

### BACKGROUND OF THE INVENTION

1. Field of Invention

This invention relates generally to a method of making, e.g., mass-producing, disposable absorbent articles, e.g., diapers, and more particularly to a method of mass-producing disposable absorbent articles which exhibit consistent absorbency characteristics and the disposable articles produced by the method.

2. Description of Related Art

Disposable absorbent articles, e.g., diapers, protective underwear, panty liners, bladder control pads, shields, belted undergarments, guards, etc., frequently make use of a liquid absorbent core located between a top-sheet and a back sheet. The top-sheet is commonly formed of a material which is pervious to body fluids, e.g., urine, to promote the transfer of such fluids into the core with minimal fluid retention by the top-sheet. The back-sheet is commonly formed of a liquid impervious or hydrophobic material to form a barrier wall so that any fluid absorbed by the article cannot escape out the back-sheet. In many cases, a fluid “acquisition,” “surge” or “transfer” layer is located between the top-sheet and the core to facilitate the transference of body fluid(s) into the core.

In some applications the top-sheet of the disposable absorbent article is formed of a woven, non-woven or carded fibrous web. In other cases a perforated or apertured polymeric film is used in place of the fibrous web top-sheet. Typically most of such apertured films are of a three dimensional nature, e.g., they are embossed and/or debossed to include numerous protuberances, channels, capillaries, hills and valleys, etc. Such three dimensional apertured film top-sheets exhibit a pleasing soft, cloth-like appearance similar to that of the prior art woven, non-woven or carded fibrous webs, while providing for a quick and efficient transfer of liquid therethrough to the underlying absorbent layer(s).

Examples of perforated three dimensional film top-sheets are found in U.S. Pat. Nos.: 3,929,135 (Thompson), 4,324,246 (Mullane et al.), 4,327,730 (Sorensen), 4,463,045 (Ahr et al), 4,552,709 (Koger et al.), 4,601,868 (Radel), 4,609,518 (Curro et al.), 4,629,643 (Curro et al.), 4,690,679 (Mattingly, III et al.), 4,806,411 (Mattingly, III et al.), 5,514,105 (Goodman et al.), D362,120 (Suskind et al), and D364,040 (Suskind), and in United States Statutory Invention Registration H1575 (Daugherty et al.).

The absorbent core of many disposable diapers and other higher performance incontinence products, e.g., adult briefs, typically have cores with enhanced absorbency capability. This enhanced absorbency can be accomplished by use of an air-laid super absorbent material, or by the inclusion of absorbency enhancers, e.g., materials sometimes referred to as “super-absorbent-polymers” (which may be in the form of particles or fibers) with other absorbent materials, such as a fluff, e.g., comminuted wood pulp or other cellulosic fibers. Examples of super absorbent materials are hydrogel polymer particulates, sometimes referred to as “SAP,” and hydrogel polymer fibers, sometimes referred to as “SAF.” The fluff for the core serves as a means for quickly absorbing the liquid transferred to it. Unfortunately, while fluff is suitable for the task of quick absorption it is somewhat deficient from the standpoint of liquid retention and ability to accommodate repeated insults. SAP on the other hand, has the ability to retain absorbed liquid and to handle repeated insults. The drawback with SAP and SAF are their slowness in absorbing liquid. Accordingly, heretofore disposable absorbent articles have typically taken two approaches to ensure efficient action. In particular, they have included a higher ratio of SAP to fluff to facilitate the faster rate of absorption of the liquid by the fluff of the core until the SAP can absorb and trap it, and have also included an the heretofore mentioned acquisition or surge layer (also sometimes called a “transfer” layer) disposed over the core. Acquisition layers have typically been constructed of a woven, non-woven or carded fibrous material. They are arranged to quickly absorb the liquid through the absorbent article's cover stock, liner, or top-sheet for temporary retention (e.g., to act as a temporary reservoir), and to transfer that liquid into the underlying core at a rate at which the core can absorb for final or permanent retention. In particular, an acquisition layer improves “wicking” of the absorbent article by spreading the body fluid in the “x” and “y” plane over the area of the core encompassed by the acquisition layer while also carrying the fluid in the “z” direction to the core.

Examples of prior art acquisition layers are disclosed in U.S. Pat. Nos. 4,988,344 (Reising et al.), 4,994,037 (Bernardin), 5,294,478 (Wanek et al.); 5,300,054 (Feist et al.), 5,304,161 (Noel et al.), 5,387,208 (Ashton et al.), 5,460,622 (Dragoo et al.), 5,486,167 (Dragoo et al.), 5,520,673 (Yarbrough et al.), 5,522,809 (Larsonneur), 5,558,655 (Jezzi et al.), 5,591,149 (Cree et al.), 5,607,414 (Richards et al.), 5,609,588 (DiPalma et al.), 5,730,737 (Widlund et al.), 5,752,945 (Mosley et al.), 5,833,678 (Ashton et al.), 5,843,055 (Seger), 5,855,572 (Schmidt), 5,895,379 (Litchholt et al.), and 5,906,602 (Weber et al.). Examples of commercially available materials used for acquisition layers in disposable absorbent articles are through-air bonded staple fibers, adhesively bonded staple fibers, and thermally point bonded staple fibers.

As will be appreciated by those skilled in the art increasing the SAP (or other absorption enhancing material) to fluff ratio to provide an absorbent product, e.g., diaper, suitable for accommodating multiple insults of body fluids would concomitantly place an additional burden on the liquid acquisition layer to facilitate transfer of the liquid at a rate that the SAP/other absorption enhancing material could accommodate. Heretofore the materials making up the prior art liquid acquisition layers have provided less than desired results.

Various absorbent articles which are commercially available have made use of various layers of materials. For example, sanitary pads made by Tyco Healthcare Retail Group, Inc. and sold under the trademark EVERYDAY PANTILINER have included a top sheet formed of a fibrous material over the marginal edges of the pad, but not over the intake or “target” zone (i.e., the area at which the body fluid(s) gain(s) ingress into the absorbent article), a three dimensional apertured film forming the top layer of the intake/target zone, a fluid acquisition layer formed of a fibrous material (non-woven) web, and a core formed of an air laid web containing super absorbent material. Other pads in the form of Ultra Thins are also made by Tyco Healthcare Retail Group, Inc. and sold under trademark FRESH TIMES have included a top sheet formed of an apertured film, an air-laid acquisition layer and a core formed of a combination of air-laid and SAP or an air laid super absorbent material. Still other pads in the form of Contour Maxi pads are made by Tyco Healthcare Retail Group, Inc. and sold under the trademark FRESH TIMES have included a top sheet formed of an apertured film and a core formed fluff. Still other pads in the form of Maxi pads are made by Tyco Healthcare Retail Group, Inc. and sold under the trademark FRESH TIMES have included a top sheet formed of a fibrous material, a tissue fluid acquisition layer and a core formed of fluff. Diapers made by Tyco Healthcare Retail Group, Inc. and sold under the trademark HAPPIES have included a non-woven top sheet, a through-air-bonded (or non-woven) acquisition layer, a tissue layer, and an absorbent core made of fluff and SAP.

In U.S. Pat. No. 6,455,753 (Glaug et al.), assigned to Tyco Healthcare Retail Group, Inc., the same assignee as this invention, and whose disclosure is incorporated by reference herein there is disclosed a disposable absorbent article, e.g., a diaper, arranged to be worn by a wearer to trap and collect fluid waste products, e.g., urine, of the wearer. That article successfully addressed a need that had existed for a diaper or other high capacity absorbent article making use of a relatively high SAP/other absorption enhancing material content core and an effective liquid acquisition system to transfer the liquid into the core. To that end, the article of the Glaug et al. patent has a flexible chassis and plural tabs for holding the diaper in place on the wearer. The absorbent system of the product is made up of a top-sheet, a fluid acquisition system, and a fluid absorbent core. The top sheet is formed of a fluid pervious material, e.g., a fibrous material. The core includes fast acting fluid absorbing material, e.g., fluff, and a slow acting fluid absorbing and retaining material, e.g., SAP. The fluid acquisition system comprises a first fluid acquisition layer formed of apertured polymeric, e.g., three dimensional, film and a second fluid acquisition layer, e.g., a fibrous material, secured together and located between the top-sheet and the core, with the first fluid acquisition layer facing the top-sheet and the second fluid acquisition layer facing the core. The fluid acquisition system serves to facilitate the transference of fluid into the core, e.g., spread out the fluid over the core and providing it to the core at a rate at which the materials of the core can accommodate.

While all of the foregoing disposable absorbent articles are suitable for their intended purposes, they never the less leave something to be desired from the standpoint of manufacturing them on a mass-production basis so that they exhibit a consistency of absorbency and “rewet” characteristics, particularly those articles making use of pulp cores with or without superabsorbent particles or fibers.

### BRIEF SUMMARY OF THE INVENTION

In accordance with one aspect of this invention there is provided a method of making, e.g., mass-producing, disposable absorbent articles, e.g., diapers, exhibiting consistent fluid absorption characteristics. Each of the articles is arranged to be worn by a wearer to trap and collect fluid waste products of the wearer. The method basically comprises providing a top-sheet and a fluid absorbent core. The top sheet is formed of a fluid pervious material and has a fluid intake zone. The fluid absorbent core comprises a fluid absorbing pulp material, e.g., fluff and superabsorbent particles or fibers. A composite fluid acquisition system is also provided as part of the method. The composite fluid acquisition system comprises a first fluid acquisition layer and a fluid absorbency enhancing layer. The first fluid acquisition layer comprises an apertured film. The fluid absorbency enhancing layer comprises an air-laid substrate. The first acquisition layer is located, e.g., placed, under the top-sheet and over the fluid absorbency enhancing layer at the fluid intake zone during the process of making each absorbent article.

In accordance with another aspect of the method of this invention the composite fluid acquisition system additionally comprises a second fluid acquisition layer. The second fluid acquisition layer comprising a fibrous, fluid pervious material. The second acquisition layer is located, e.g., placed, under the first fluid acquisition layer at the fluid intake zone during the process of making each absorbent article.

In accordance with still another aspect of this invention the composite fluid acquisition system is cut from materials forming its various layers to a predetermined profile and is then placed over the core and under said top sheet at the fluid intake zone.

In accordance with yet another aspect of this invention the fluid absorbency enhancing layer additionally comprises super absorbent particles or fibers and a binder.

Another aspect of this invention constitutes a mass-produced disposable absorbent article e.g., a diaper, exhibiting consistent fluid absorption characteristics. That article is arranged to be worn by a wearer to trap and collect fluid waste products of the wearer and comprises a top-sheet and a fluid absorbent core. The top sheet is formed of a fluid pervious material and has a fluid intake zone. The absorbent core comprises a fluid absorbing pulp material, e.g., fluff and superabsorbent particles or fibers. The composite fluid acquisition system comprises a first fluid acquisition layer and a fluid absorbency enhancing layer. The first fluid acquisition layer comprises an apertured film, e.g., a three dimensional apertured film. The fluid absorbency enhancing layer comprises an air-laid substrate, which may also contain super absorbent particles or fibers and a binder. The first acquisition layer is located under the top-sheet and over the fluid absorbency enhancing layer at the fluid intake zone.

In accordance with another aspect this invention the composite fluid acquisition system of the article additionally comprises a second fluid acquisition layer. The second fluid acquisition layer comprising a fibrous, fluid pervious material. The second acquisition layer is located under the first fluid acquisition layer at the fluid intake zone.

In accordance with still another aspect this invention the composite fluid acquisition system of the article additionally comprises a carrier layer comprising tissue. The carrier layer is located under the fluid absorbency enhancing layer at the fluid intake zone and over the absorbent core.

In accordance with yet another aspect of this invention the fluid absorbency enhancing layer additionally comprises super absorbent particles or fibers and a binder.

### BRIEF DESCRIPTION OF SEVERAL VIEWS OF THE DRAWINGS

The invention will be described in conjunction with the following drawings in which like reference numerals designate like elements and wherein:

FIG. 1 is an isometric view of one preferred embodiment of the subject invention, e.g., a diaper;

FIG. 2 is an enlarged, exploded sectional view taken along line 2-2 of FIG. 1;

FIG. 2A is a sectional view, like that of FIG. 2, but showing an alternative embodiment of a portion of the diaper of FIG. 1;

FIG. 3 is a sectional view, like that of FIGS. 2 and 2A, but showing another alternative embodiment of a portion of the diaper of FIG. 1; and

FIG. 3A is a sectional view, like that of FIGS. 2, 2A and 3, but showing still another alternative embodiment of a portion of the diaper of FIG. 1.

### DETAILED DESCRIPTION OF THE INVENTION

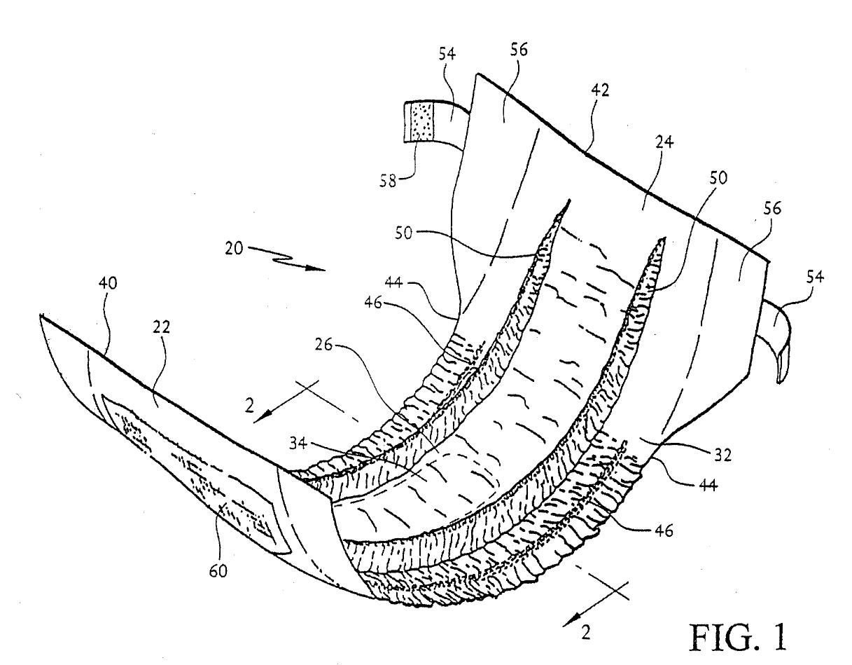

Referring now to the various figures of the drawing wherein like reference characters refer to like parts, there is shown in FIG. 1 a disposable absorbent article 20 constructed in accordance with one embodiment of this invention. It should be pointed out that as used herein the term “disposable” means that article is designed to be used until soiled, either by urination or otherwise, and then discarded, rather than being washed and used again.

In the embodiment of FIG. 1 the article 20 is in the form of a diaper. While the following description will focus on diapers, it should be clear that the subject invention can be used for any type of absorbent article or garment to be worn by a person for trapping urine or menses. Examples of such other articles are adult briefs or other incontinent products, pads, etc. Moreover, a portion of the diaper 20, making up its composite fluid acquisition system to be described later, can itself form the absorbent article.

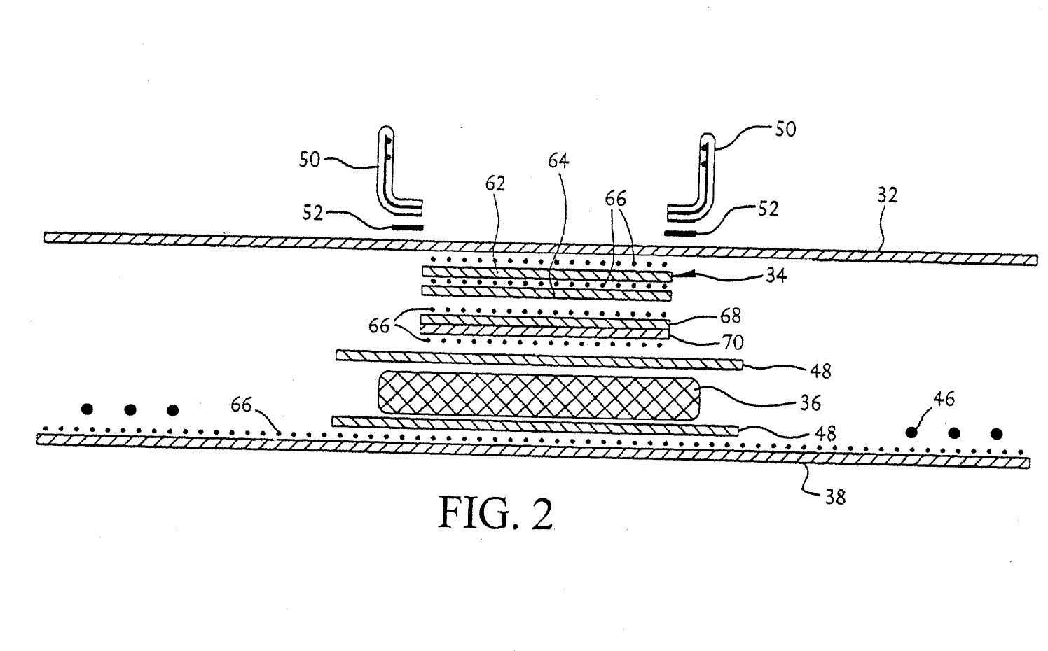

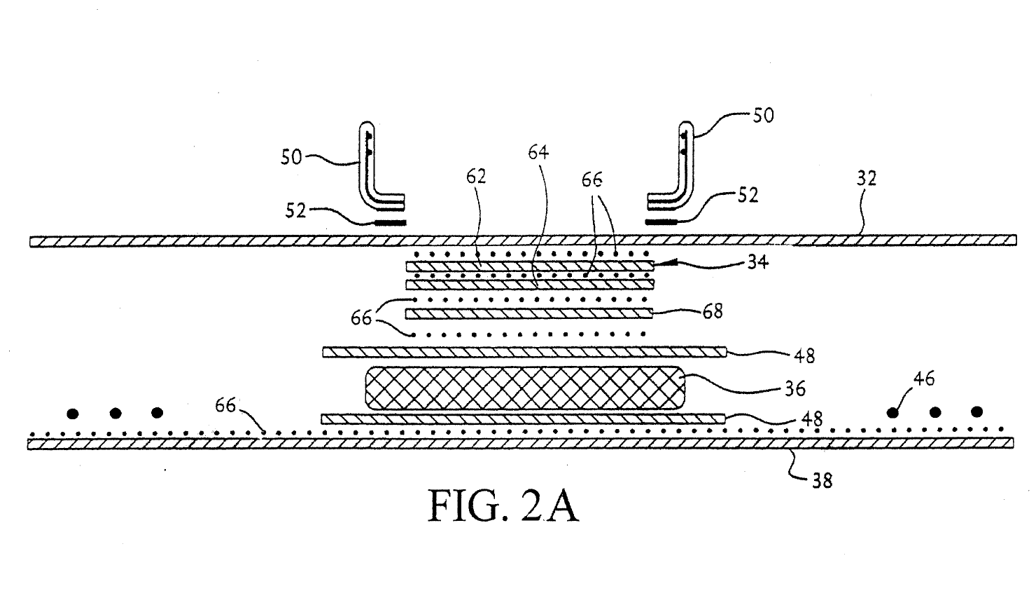

As best seen in FIG. 1, the exemplary diaper 20 basically comprises a chassis including a front waist portion 22, a back waist portion 24, and a crotch portion 26 and is of generally conventional construction, except for the inclusion of a composite fluid acquisition system (to be described later) for effectively transferring received body fluid(s) to an absorbent core capable of handling repeated insults of the fluid(s). In the exemplary embodiment to be described hereinafter the core may include a higher proportion of liquid-absorption-enhancing materials, e.g., SAP or SAF, in its absorbent core than commonly used in the prior art. The components of the composite fluid acquisition system of the diaper 20 will be described in detail later. Prior to describing those components a brief description of the other, and conventional, portions of the diaper will now be discussed. To that end and as best seen in FIG. 2, the diaper 20 basically comprises a body-side liner or top-sheet 32, the heretofore mentioned composite fluid acquisition system 34, a liquid absorbent structure or core 36 including liquid-absorption-enhancing materials, e.g., SAP, etc. (to be described later), and an outer cover or back-sheet 38.

The top-sheet 32 is arranged to face toward the body of the user, when the diaper is in place, with the back-sheet facing away from the wearer. The top-sheet is superimposed over the back-sheet, with the absorbent core 36 interposed therebetween. The composite fluid-acquisition system 34 is located on top of the core 36 and under the top-sheet 32 to facilitate the passage of liquid waste into the core for absorption thereby. The top-sheet 32 and/or back-sheet 38 can be any suitable shape and dimensions for other designs or constructions, as will be clear from the other embodiments disclosed herein.

The back-sheet 38 comprises front edge 40, a back edge 42, and a pair of side edges 44. Each side edge includes a central, cut-out to define a respective leg cut out. The crotch portion 26 of the diaper is located between the leg cut-outs.

The top-sheet 32 may be of the same shape as the back-sheet 38 or of a different shape and is bonded to the back-sheet 36 around its entire periphery, with the absorbent core 36 and the composite fluid acquisition system 34 interposed therebetween. The back-sheet and top-sheet can be joined together in any suitable manner, e.g, by adhesive bonding. The adhesives can be applied in any manner such as by spraying, slot-coat extrusion, printing, or the like. The applied adhesive can be in any desired configuration or design, such as continuous or discontinuous beads, continuous or discontinuous swirls, meltblown patterns, spray patterns, or the like. Alternatively, the joining of layers and structures can be accomplished by heat sealing, ultrasonic bonding, or the like.

Each lateral side edge 44 of the diaper 20 is elasticized by means of plural, e.g., three, longitudinally extending elastic, e.g., LYCRA 940 decitex, threads or strands 46 disposed along the length of the cut away portion of that side edge. The strands may be attained from E.l. DuPont de Nemours and Company, Wilmington, Del., and are held in place by a suitable elastic adhesive, such as that used to hold the elastic foam of the waist portion in place. The elastic adhesive is intermittently applied along the top sheet to allow the diaper to be actively stretchable along the leg cut outs and not all the way to the edges of the respective waist portions, thereby enable the diaper to closely conform about the legs of the wearer for impeding the egress of waste material from the crotch region, as is conventional. Other arrangements can be used to elasticize the sides of the crotch portion of the diaper. For example, in lieu of plural longitudinally extending elastic threads 46, multiple strands of elastic material can be arranged in other orientations, intersecting, diagonal, or any combination thereof, or can be a film or laminate of various types of elastomeric material.

The back-sheet 38 or cover is preferably formed of a laminated sheet of a non-woven material and film (with the non-woven side positioned as the outermost layer). Such material should be hydrophobic, soft in texture, and strong in tensile strength. One particularly suitable material is a spunbond-meltblown-spunbond (SMS) web having a basis weight of about 15 gsm per square meter (gsm), available from AVGOL Nonwoven Industries LTD., Holon, Israel. The spunbond layer is made of polypropylene fibers. Such composites provide the dual advantages of liquid barrier properties of film along with a soft, warm outer fabric texture. The non-woven outer cover can also be made of other suitable cloth-like materials, e.g., spun-bond or thermal-bond non-woven web made of either polypropylene, polyethylene, polyester, bi-component fibers (polyethylene/polypropylene or polyethylene/polyester), or any combinations of these fibers. Various multiple layer configurations or fiber denier variations may be used. Another example includes hydro-entangled non-woven webs, which may contain some cotton and/or rayon fibers blending in with thermal-plastic fibers. Cellulose fibers can also be blended in at small percentages to reduce cost. Still another example is a non-woven outer-cover made of stretchable or elastic materials, such as elastomeric composites of non-woven(s) and elastic membranes or a single layer of elastic material. The elastomeric composite can comprise of an inner layer of pre-stretched extruded elastic film sandwiched between and attached to a pair of non-woven webs. The non-woven webs may consist of spun-bond web, thermal-bond web, or a combination of the two. Preferably, the elastic film is made of synthetic rubber and the non-woven made of spun-bond polypropylene.

Other materials for forming the back-sheet 38 may include polypropylene films, co-extruded films (polyethylene and ethylene vinyl acetate), co-polymer films (polyethylene/polypropylene), and polylaminates (polypropylene nonwoven and polyethylene film). Still another example is a film made of a “breathable microporous’ polyethylene. Suitable breathable films are available from Pliant Corporation, Williamsburg, Va. This material allows water vapor to pass through it over time, while being impervious to liquid water. The water vapor transmission rate may range from 200-2000 grams per square meter per 24-hour period.

The composite fluid-acquisition system 34 will be described in considerable detail later. Suffice it for now to state that it includes at least two layers of materials and is constructed such that the layers cooperate to manage, transport, accommodate and/or direct high volumes and high flow rates of urine or other body fluid received from the top sheet target zone into the absorbent core 36 at a rate that the core can handle, despite multiple insults of such fluid. Moreover, the combination of the composite fluid acquisition system 34 and the core 36 results in an absorbent article that exhibits consistent absorbency and rewet characteristics even though it is mass produced. To that end, as shown in FIGS. 1, 2, 2A, 3 and 3A, one of the layers of the composite fluid acquisition system 34 acts to enhance the absorbency consistency of the article 20, notwithstanding that the absorbency of the core 36 may vary or be inconsistent from article to article as a result of the core's manufacturing process and constituents.

In order to enable urine to quickly and efficiently pass through the top-sheet and into the underlying acquisition system 34 for subsequent transference to the absorbent core 36 for trapping therein, the top-sheet 32 is preferably liquid permeable. In particular, the top sheet 32 may be selected from a variety of textile-like films and fabrics. Suitable fabrics include non-woven materials that are pervious to liquid, soft and pliable. Preferred non-woven materials include spun-bonded polypropylene; spunbonded polyethylene; thermally bonded webs of staple fibers preferably polypropylene shape or sheath/core bi-component fibers having a core of polyester or polypropylene and a sheath of polyethylene. To enhance the fluid control properties of the aforementioned liners, surfactants or wetting agents typified by Silastol PST available from Schill & Seilacher GmbH & Co., Boblingen, Germany and may be applied to the fluid receiving zones of the liner selectively having the outer zones untreated to reduce migration excreted fluid such as urine into the outer diaper regions leading to diaper leakage.

If desired, the top sheet 32 may be formed of a liquid impermeable material having plural apertures or pores extending therethrough so as to make the material liquid permeable.

The absorbent core 36 preferably is a uniform basis-weight rectangular member which is centered in the diaper and extends from close to the front waist edge to close to the back waist edge. The core can be made up of any suitable absorbent material, as well as combinations of different types of absorbent material(s). For example, in the preferred embodiment of FIG. 1 the absorbent core 36 is formed of a mixture of pulp fluff and SAP wrapped in a liquid permeable tissue wrap 48 (only two sheets of which are shown in FIG. 2). Examples of SAP include polyacrylamides, polyvinyl alcohol, polyacrylates, various grafted starches, and the like. A desired super absorbent material is a cross-linked polysodium acrylate, which can be purchased from Chemdal Corporation, Palatine, Illinois, under the trademark ASAP 2260. The super absorbent materials can be in various geometric forms, such as various shaped particles, fibers, foams, and layers. The fluff and SAP are present in a ratio of about 11.5 grams of SAP to 15.5 gms of fluff for a size 4 diaper, and have a core density range of about 0.12 to 0.20 grams per cubic centimeter. Other SAP materials can be used, such as those sold under the trade designations IM-3900, #2260 and #7710, by BASF Aktiengesellschaft of Ludwigshafen, Germany.

Moreover, the core 36 can be of any shape and can be a single, integral absorbent structure, or can comprise a plurality of individual separate absorbent structures and/or absorbent materials that are operably assembled together. It can also consist of air-laid non-woven web that contains super-absorbent particles and/or super-absorbent fibers, polymeric binder and cellulose pulp fibers. In one exemplary embodiment shown in FIGS. 2 and 2A the absorbent core 36 is sandwiched between two plies of tissue, is aligned on top of the back-sheet and adhered down with construction adhesive. The tissue has a basis weight of 16.3-17.1 gsm. Suitable tissues are available from Cellu Tissue Corporation, East Hartford, Connecticut. The absorbent core is centered along the transverse direction and registered in the machine (longitudinal) direction within the diaper's chassis.

The amount of each absorbent material and SAP/fluff ratio depends on the size of the brief, e.g., “Small”, “Medium”, “Large” or “Extra Large” and the construction of the liquid acquisition or transfer system 34.

The diaper 20 also includes a pair of conventional “standing leg gathers” or cuffs 50 or liquid-impervious gaskets to provide leakage control in the crotch region. The standing leg gathers are located so that they extend along the leg opening region of the diaper as disclosed in U.S. Pat. No. 4,695,278 (Lawson) and U.S. Pat. No. 4,795,454 (Dragoo), both of which are incorporated by reference herein. Each standing leg gather is elasticized and extends from the edge of the front waist portion to the edge of the rear waist portion and along a respective side marginal edges of the core 36 and upstanding from the top-sheet 32. The standing leg gathers are secured in place by a suitable adhesive, e.g., construction adhesive 52.

The diaper 20 is arranged to be held in place on the body of the wearer in a conventional manner, e.g., by means of a pair of fastening tabs or tapes 54 projecting outward from a pair of respective ear portions 56 forming the side edges of top sheet 32 of the diaper contiguous with its back waist portion 42. In particular, each tab 54 includes a patch 58 of a myriad of small hooks on its underside surface. Each patch is arranged to be releasably secured to a “landing zone” portion 60 on the outer cover in the front waist region of the diaper. The landing zone is located at a position so that when the diaper is folded in half with the front waist portion disposed opposite the back waist portion, the landing zone 60 will be aligned with the tabs 54.

The landing zone 60 basically comprises a rectangular panel of whose outer surface comprises a myriad of small loops arranged to be engaged by the small hooks of the patch 58 of each fastening tab.

When the diaper is in place on the person with the front waist portion disposed over the lower abdomen, the back waist portion disposed over the lower back and buttocks region, and the crotch portion between the legs, each tab 54 may be brought into engagement with the a portion of the landing zone 60 closest to that tab on the front portion of the diaper so that the myriad of hooks on the patch engage the myriad of loops of the landing zone 60 to releasably secure the tab thereto. Any suitable multi-hook and multi-loop materials may be used. Particularly suitable multi-hook patches 54 are available from Gottlieb Binder GmbH & Company, Holzgerlingen, Germany, under the model designation as Microplast, while a particularly suitable multiloop material is a polyester fiber material having a basis weight of 40 gsm with a laminated polypropylene film (0.75 mil.) backing is available from Avery Dennison, Painesville, Ohio.

Alternatively the tabs 54 may be in the form of adhesive tapes, such as #4424 available from Avery-Dennison, Mentor, Ohio, and the landing zone may be formed of a polypropylene film with a pre-applied adhesive in a selected print pattern, such as #KN-6977 also available from Avery-Dennison, Mentor, Ohio.

The composite fluid-acquisition system 34 of the exemplary embodiments of FIGS. 1, 2, and 3 basically comprises four layers disposed over one another. In particular, the uppermost of the layers making up the composite fluid acquisition system 34 comprises an apertured polymeric film 62, which is preferably three dimensional in nature, e.g., is embossed and/or debossed, like that used heretofore and described above for cover sheets of diapers. Examples of suitable three dimensional apertured film materials are available from Tredegar Film Products of Richmond, Va. under the following trade designations which are listing in increasing size of their apertures from smallest to largest: 40 HEX, 25 HEX, ROCC22 and 22 HEX. These films exhibit the following porosities: 40 HEX having a porosity of 110 m3/min/m2); 25 HEX having a porosity of 11 m3/min/m2; ROCC22 having a porosity of 85 m3/min/m2. Other materials can be used such as a polyethylene film apertured like that shown in U.S. Pat. Design No. D362,120 (Suskind et al.), whose disclosure is incorporated by reference herein, or other three dimensional polymeric apertured films, such as those in the patents discussed above. In fact, for some applications two dimensional apertured films, like those of the prior art, may be used for the layer 62.

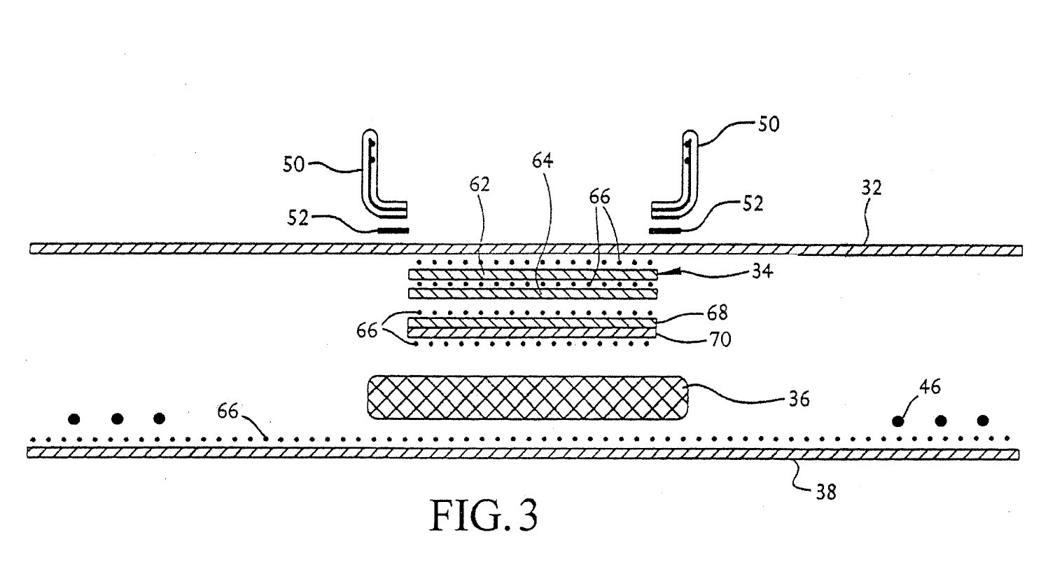

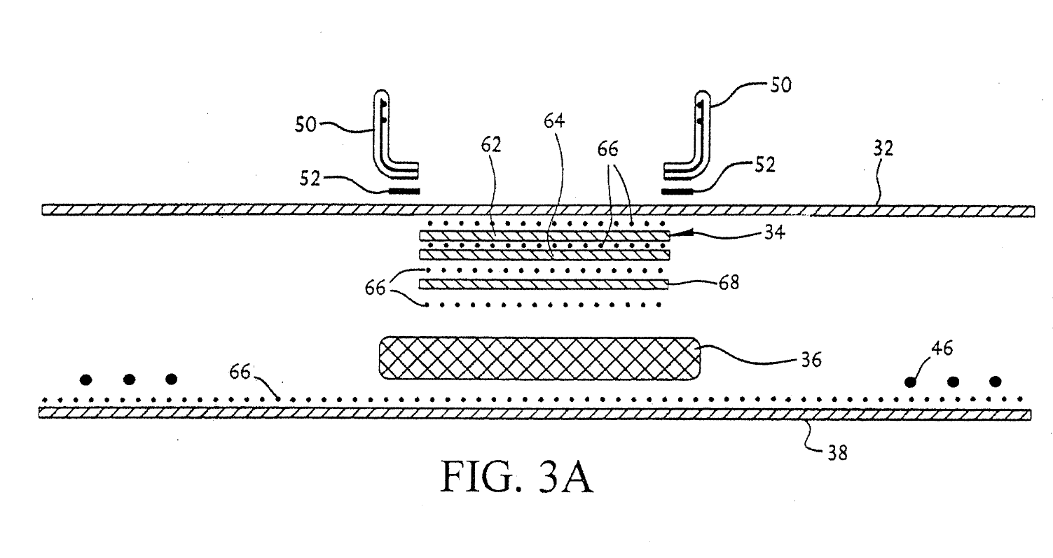

If the apertured film has small apertures it is preferable to make use of an underlying layer 64 of a nonwoven material in order to be able to hold and slowly transfer fluid into the core after multiple insults. In particular, when the first void of fluid (e.g., urine) is emitted upon an absorbent article having an absorbent core, particularly with fluid retaining materials (e.g., SAP), the core becomes saturated and does not take in repeated voids as rapidly as the first time. Thus, some material(s) with “reservoir” space is/are needed in addition to the core, in order to handle the multiple insults. A nonwoven layer under the apertured film serves that purpose. Such an arrangement is shown in the exemplary embodiment of FIGS. 2 and 3. The material making up the layer 64 can be any type of fibrous material, e.g., a through-air bonded/carded web, a spun-bond bi-component non-woven web, a web of cross-linked cellulosic fibers. One particular suitable material is Adhesive-Bonded Polyester (ABPET) available from PGI Nonwovens, Landisville, N.J., and has an overall basis weight of 40 gsm. Other materials can be used such as Through Air-Bonded Bi-Component Fiber (TABICO), Thermal Bonded Polypropylene (TBPP), Chemical Treated Mechanical Pulp (CTMP), Cellulose Fibers cross linked with Citric Acid (Curly “Q” Cellulose Fibers), and Spunbond Polypropylene (SBPP).

The layers 62 and 64 are preferably, but not mandatorily, adhered or secured together by any suitable adhesive 66 (e.g., a construction adhesive or hydrophillic adhesive, such as Cycloflex adhesive available from National Starch and Chemical, Bridgewater, N.J.). The layers 62 and 64 can also be joined or bonded together by various other methods, such as ultrasonic bonding, heat sealing, hot knife slitting, hydroentanglement, physical stitching or sewing, etc., or any other suitable technique known in the art.

If the apertured film layer 62 has apertures whose size is large enough to hold enough fluid to act as a reservoir, the nonwoven layer 64 may be omitted. Such an embodiment is shown in FIGS. 2A and 3A, where the composite fluid acquisition system only includes the apertured film layer 62. Examples of large diameter three-dimensional apertured films suitable for applications like the embodiments of FIGS. 2A and 3A are also sold by Tredegar Film Products under the trade designations AQUIDRY X-28617 (having a porosity of 170 m3/min/m2)b and X-28618 (having a porosity of 175 m3/min/m2).

As best seen in FIGS. 2, 2A, 3 and 3A, another layer 68 is disposed immediately below the nonwoven layer 64 (if that layer is used) or under apertured film layer 62 (if the nonwoven layer 64 isn't used). The layer 68 forms the heretofore mentioned fluid absorbency enhancing layer and is preferably coextensive in size with the layers 62 and 64, but may be or greater or smaller in width and/or length if desired. The layer 68 is interposed between either the layer 64 and the core 36 or layer 62 and the core (depending if layer 64 is used or not). The layer 68 preferably comprises an air-laid material substrate. In accordance with a preferred aspect of this invention the air-laid substrate contains super absorbent particles or fibers (24.5%), cellulose pulp fibers (61.2%), latex binder (0.6%), bi-component fibers (6.9%) and carrier tissue (6.8%) and a binder to form a composite, but the use of such added materials is optional.

One particularly effective air-laid composite material is sold by Buckeye Nonwovens Division of Memphis, Tenn. under the trade designation of BUCKEYE VISORB 3905 and comprises a 100-300 gsm pulp/SAP/binder composite. Other suitable materials that can be used are BUCKEYE DX261, DX262, DX291, 8902 and 3008 also available from Buckeye Nonwovens Divisions. Air-laid composites from Rayonier Performance Fibers of Jessup, Ga. sold under the trade designations J2500740, J3500740 and J4000755 may also be used, as can the composite sold by Concert GmbH of Falkenhagen, Germany under the trade designation 300.0061.

The air-laid layer 68 is preferably positioned at the urine void or target area. When so positioned it acts as a three-dimensional absorbent core pocket on top of the uniform or profiled basis weight core 36, as will be described later.

As should be appreciated by those skilled in the art, the use of the air-laid layer between the acquisition layer(s) 62/64 and the core 36 will result in more consistent absorbency performance (e.g., rewet, absorbency rate, etc.), since air-laid composite materials can be controlled better in blending the pulp/SAP/binder of the composite as compared to absorbent cores made by conventional hammer mill systems. In short, there is considerable variability in absorbency characteristics from core to core since there are likely to be variations in the amount of pulp/SAP/binder incorporated from core to core in mass produced cores. The use of the air-laid composite layer provides a place for effective and consistent fluid absorbency to effectively smooth or even out the variability of absorbency from core to core in mass produced cores used in the diaper 20. This results in a mass produced diaper 20, or any other absorbent article incorporating the subject invention, that provides more consistent and uniform absorbency characteristics than the prior art.

The air-laid composite layer 68 may include a tissue layer 70 disposed beneath it, such as shown in FIG. 2 and FIG. 3. Such an arrangement is advantageous when the air-laid layer 68 includes SAP to deter the SAP from falling out of it during fabrication and assembly of the diaper 20 (or any other absorbent article the composite fluid acquisition system 34 is used in). If little or no SAP is used in the air-laid layer 68 the tissue layer 70 may be eliminated. Such an arrangement is shown in the embodiment of FIG. 2A and 3A.

As best seen in FIGS. 2 and 3, the composite fluid-acquisition system 34 is preferably adhesively secured in place by any suitable adhesive 66 (e.g., a construction adhesive or hydrophillic adhesive, such as Cycloflex adhesive available from National Starch and Chemical, Bridgewater, N.J.). In particular, the acquisition system 34 is secured in place by use of an adhesive 66 between its upper layer 62 and the top sheet 32. The lower layer 64 is secured to the air-laid composite layer 68 by an adhesive 66, and the tissue layer 70 is secured to the tissue covering or wrap 48 of the core 36 by an adhesive 66.

In the exemplary embodiment described above, 14 grams of fluff and 10 grams of SAP is used for the absorbent core for both the “Medium” and “Large” sizes of diapers, with a transfer or acquisition system including an upper layer 62 of 36.6 gsm, and a lower layer 64, of 250 gsm and approximately 70 mm wide by 165 mm long through air bonded bicomponent fibers adhered to the air-laid composite layer 68. That layer is disposed on top of the absorbent core in a “continuous” configuration.

The embodiments shown in FIGS. 3 and 3A are identical to the embodiment of FIGS. 2 and 2A, respectively, except that no tissue cover or wrap 48 is used over the core. Such an arrangement is suitable where the core has a lower amount of SAP as compared to the amount of pulp in it, e.g., 85% pulp and 15% SAP, and can be used for incontinent products, such as adult briefs. Where, however, the core has a higher proportion of SAP, such as 55% pulp and 45% SAP, such as the case with children's diapers, the use of a tissue wrap 48 is desirable to prevent migration of the SAP from the core.

In order to facilitate the transference of the body fluid(s) through the absorbent article into the core 36, one or more of the various layers may be treated with any suitable surfactant(s), such as a “fast finish” surfactant for the layer 64 and monoglyceride and diglyceride for the layer 62. In the exemplary embodiment the layer 62 treated with a suitable surfactant is available from Tredegar Film Products of Terra Haute, Ind. as X-6958 white C120 apertured film. It should be pointed out at this juncture that in the exemplary embodiment of the diaper shown and described herein both the layers 62 and 64 are treated with surfactants, but either or neither layer may be so treated.

As best seen in FIG. 1 the composite acquisition system 34 is located in the diaper over the core in the front portion of the diaper making up the fluid intake or target zone of the diaper, i.e., the portion of the diaper to directly receive the liquid body waste(s) from the wearer. If desired the acquisition system 34 can be extended into the rear portion of the diaper, as well.

As should be appreciated by those skilled in the art, while various layers of the diaper 20 have been shown in the drawings as including adhesive layers therebetween, such an arrangement is not mandatory. Thus, one or more of the adhesive layers 66 can be eliminated, if desired.

The absorbent article 20 operates as follows upon the receipt of a fluid (e.g., urine) insult to its top sheet 32 intake zone. The fluid penetrates the top sheet 32 and passes through the apertured polymeric layer (film) 62 of the fluid acquisition system generally perpendicularly to the layer 62. Fluid flow is faster into the diaper 20, i.e., through the top sheet into the diaper, than coming out of the diaper through the top sheet. This is due to the fact that the apertured polymeric film 62 of the exemplary preferred embodiment has three dimensionally shaped apertures directed inwardly towards the core 36. In particular, with the preferred exemplary embodiment the apertured film 62 includes a myriad of very tiny conically shaped apertures, which taper in the direction from the top sheet 32 towards the core 36. This arrangement results in a faster and higher fluid volume intake into the diaper and a slower and lower fluid volume output after the diaper has been saturated with fluid and exposed to compressive forces. The fluid then contacts the layer 64 of the composite fluid acquisition system 34, where the fluid experiences some lateral distribution both transversely and longitudinally in the plane making up the layer 64 due to the fiber orientation of that layer in the machine direction. From there the fluid enters into the fluid absorbency enhancing layer 68 where some of it is spread out and absorbed and the remainder passes into the core 36 for ultimate absorption and retention.

The composite fluid acquisition system 34 of this invention optimizes the fluid penetration rate and volume into the absorbent core 36 below it, while also minimizing the fluid from exiting the diaper 20 when it is saturated and under high stress. Moreover, the air-laid fluid absorbency enhancing layer serves to act as a controlled reservoir for some of the fluid, thereby rendering the combination of the fluid acquisition system and the core much more consistent insofar as fluid absorption and rewet characteristics are concerned. The result is a diaper which consistently exhibits high fluid intake and low fluid rewet characteristics, features which are desirable to obtain high absorbency performance with minimal fluid exposure to the wearer's skin.

It should be noted at this juncture, that the diaper 20 or any other absorbent article making use of the subject composite fluid acquisition system and a core, can be fabricated so that one can use in the fluid absorbency enhancing layer super absorbent particles or fibers exhibiting a different density from the superabsorbent particles or fibers in the core, thereby enabling one to effectively customize the absorbent article for a desired application and further enhancing its performance characteristics.

It should also be pointed out that an absorbent article, designed for small relatively capacity uses, e.g., a pad or pantiliner, can be made that only includes the composite fluid acquisition system 34, e.g., air-laid composite layer or layers 68 and either a layer 62 or a layer 62 and a layer 64, but without any core 36.

The diaper 20, or any other absorbent article contemplated by this invention, can be fabricated and assembled easily on a mass production basis. In particular, the materials making up the article's various layers can be readily shaped from sheets or webs of suitable materials. The core and the composite acquisition layer can each also be die-cut from respective suitable materials and placed at their desired positions with respect to the chassis of the article. Then the various die-cut and placed components can be assembled with suitable adhesives to complete the article. For example, the composite acquisition layer can be placed below the top sheet and at the location of the target or insult zone and over the core. This feature provides flexibility to make adjustments to that zone in terms of re-dimensioning or relocating the composite fluid acquisition layer on top of the core, e.g., the composite fluid acquisition layer can be registered more forward longitudinally in the chassis for males and more centered in the chassis for females.

While the invention has been described in detail and with reference to specific examples thereof, it will be apparent to one skilled in the art that various changes and modifications can be made therein without departing from the spirit and scope thereof.

What is claimed is:

1. A method of making disposable absorbent articles exhibiting consistent fluid absorption characteristics, each of the articles being arranged to be worn by a wearer to trap and collect fluid waste products of the wearer, said method comprising the steps of:
(A) providing a top-sheet and a fluid absorbent core, said top sheet being formed of a fluid pervious material and having a fluid intake zone, said absorbent core comprising a fluid absorbing pulp material;
(B) providing a composite fluid acquisition system comprising a first fluid acquisition layer and a fluid absorbency enhancing layer, said first fluid acquisition layer comprising an apertured film, said fluid absorbency enhancing layer comprising an air-laid substrate; and
(C) locating said first acquisition layer under said top-sheet and over said fluid absorbency enhancing layer at said fluid intake zone.

2. The method of claim 1 wherein said composite fluid acquisition system additionally comprises a second fluid acquisition layer, said second fluid acquisition layer comprising a fibrous, fluid pervious material, and wherein said method additionally comprises:
(D) locating said second acquisition layer under said first fluid acquisition layer at said fluid intake zone.

3. The method of claim 1 wherein said composite fluid acquisition system additionally comprises a carrier layer comprising tissue, and wherein said method additionally comprises:
(D) locating said carrier layer under said fluid absorbency enhancing layer at said fluid intake zone and over said absorbent core.

4. The method of claim 2 wherein said composite fluid acquisition system additionally comprises a carrier layer comprising tissue, and wherein said method additionally comprises:
(D) locating said carrier layer under said fluid absorbency enhancing layer at said fluid intake zone and over said absorbent core.

5. The method of claim 1 wherein said composite fluid acquisition system is cut from materials forming its various layers to a predetermined profile and is then placed over said core and under said top sheet at said fluid intake zone.

6. The method of claim 2 wherein said composite fluid acquisition system is cut from materials forming its various layers to a predetermined profile and is then placed over said core and under said top sheet at said fluid intake zone.

7. The method of claim 3 wherein said composite fluid acquisition system is cut from materials forming its various layers to a predetermined profile and is then placed over said core and under said top sheet at said fluid intake zone.

8. The method of claim 1 wherein said fluid absorbency enhancing layer additionally comprises super absorbent particles or fibers.

9. The method of claim 2 wherein said fluid absorbency enhancing layer additionally comprises super absorbent particles or fibers.

10. The method of claim 3 wherein said fluid absorbency enhancing layer additionally comprises super absorbent particles or fibers.

11. The method of claim 8 wherein said fluid absorbency enhancing layer additionally comprises a binder.

12. The method of claim 9 wherein said fluid absorbency consistency enhancing layer additionally comprises a binder.

13. The method of claim 10 wherein said fluid absorbency consistency enhancing layer additionally comprises a binder.

14. The method of claim 1 1 wherein said binder comprises a bicomponent fiber having an polyethylene outer sheath and a polypropylene or polyester inner core.

15. The method of claim 12 wherein said binder comprises a bicomponent fiber having an polyethylene outer sheath and a polypropylene or polyester inner core.

16. The method of claim 13 wherein said binder comprises a bicomponent fiber having an polyethylene outer sheath and a polypropylene or polyester inner core.

17. The method of claim 1 wherein said core additionally comprises super absorbent particles or fibers.

18. The method of claim 2 wherein said core additionally comprises super absorbent particles or fibers.

19. The method of claim 3 wherein said core additionally comprises super absorbent particles or fibers.

20. The method of claim 1 wherein said apertured film is three dimensional.

21. The method of claim 2 wherein said apertured film is three dimensional.

22. The method of claim 3 wherein said apertured film is three dimensional.

23. The method of claim 5 wherein said articles are arranged to be worn by a male and wherein each of said articles has front portion, a rear portion and a longitudinal central axis, said longitudinal central axis having a center located between said front portion and said rear portion, said fluid intake zone being located on said central axis in said front portion, and wherein said method additionally comprises:
(D) placing said composite fluid acquisition system over said core and under said top sheet at said fluid intake zone.

24. The method of claim 6 wherein said articles are arranged to be worn by a male and wherein each of said articles has front portion, a rear portion and a longitudinal central axis, said longitudinal central axis having a center located between said front portion and said rear portion, said fluid intake zone being located on said central axis in said front portion, and wherein said method additionally comprises:
(E) placing said composite fluid acquisition system over said core and under said top sheet at said fluid intake zone.

25. The method of claim 7 wherein said articles are arranged to be worn by a male and wherein each of said articles has front portion, a rear portion and a longitudinal central axis, said longitudinal central axis having a center located between said front portion and said rear portion, said fluid intake zone being located on said central axis in said front portion, and wherein said method additionally comprises:
(E) placing said composite fluid acquisition system over said core and under said top sheet at said fluid intake zone.

26. The method of claim 5 wherein said articles are arranged to be worn by a female and wherein each of said articles has front portion, a rear portion and a longitudinal central axis, said longitudinal central axis having a center located between said front portion and said rear portion, said fluid intake zone being located on said central axis at approximately said center, and wherein said method additionally comprises:
(D) placing said composite fluid acquisition system over said core and under said top sheet at said fluid intake zone.

27. The method of claim 6 wherein said articles are arranged to be worn by a female and wherein each of said articles has front portion, a rear portion and a longitudinal central axis, said longitudinal central axis having a center located between said front portion and said rear portion, said fluid intake zone being located on said central axis at approximately said center, and wherein said method additionally comprises:
(E) placing said composite fluid acquisition system over said core and under said top sheet at said fluid intake zone.

28. The method of claim 7 wherein said articles are arranged to be worn by a female and wherein each of said articles has front portion, a rear portion and a longitudinal central axis, said longitudinal central axis having a center located between said front portion and said rear portion, said fluid intake zone being located on said central axis at approximately said center, and wherein said method additionally comprises:
(E) placing said composite fluid acquisition system over said core and under said top sheet at said fluid intake zone.

29. A mass-produced disposable absorbent article exhibiting consistent fluid absorption characteristics, said article being arranged to be worn by a wearer to trap and collect fluid waste products of the wearer and comprising: a top-sheet and a fluid absorbent core, said top sheet being formed of a fluid pervious material and having a fluid intake zone, said absorbent core comprising a fluid absorbing pulp material, said composite fluid acquisition system comprising a first fluid acquisition layer and a fluid absorbency enhancing layer, said first fluid acquisition layer comprising an apertured film, said fluid absorbency enhancing layer comprising an air-laid substrate, said first acquisition layer being located under said top-sheet and over said fluid absorbency enhancing layer at said fluid intake zone.

30. The article of claim 29 wherein said composite fluid acquisition system additionally comprises a second fluid acquisition layer, said second fluid acquisition layer comprising a fibrous, fluid pervious material, and wherein said second acquisition layer is located under said first fluid acquisition layer at said fluid intake zone.

31. The article of claim 29 wherein said composite fluid acquisition system additionally comprises a layer comprising tissue, and wherein said tissue layer is located under said fluid absorbency enhancing layer at said fluid intake zone and over said absorbent core.

32. The article of claim 30 wherein said composite fluid acquisition system additionally comprises a layer comprising tissue, and wherein said tissue layer is located under said fluid absorbency enhancing layer at said fluid intake zone and over said absorbent core.

33. The article of claim 29 wherein said composite fluid acquisition system is of a predetermined shaped profile.

34. The article of claim 30 wherein said fluid absorbency enhancing layer additionally comprises super absorbent particles or fibers.

35. The article of claim 31 wherein said fluid absorbency enhancing layer additionally comprises super absorbent particles or fibers.

36. The article of claim 32 wherein said fluid absorbency enhancing layer additionally comprises super absorbent particles or fibers.

37. The article of claim 35 wherein said fluid absorbency enhancing layer additionally comprises a binder.

38. The article of claim 36 wherein said fluid absorbency enhancing layer additionally comprises a binder.

39. The article of claim 37 wherein said fluid absorbency enhancing layer additionally comprises a binder.

40. The article of claim 37 wherein said binder comprises a bicomponent fiber having an polyethylene outer sheath and a polypropylene or polyester inner core.

41. The article of claim 38 wherein said binder comprises a bicomponent fiber having an polyethylene outer sheath and a polypropylene or polyester inner core.

42. The article of claim 39 wherein said binder comprises a bicomponent fiber having an polyethylene outer sheath and a polypropylene or polyester inner core.

43. The article of claim 29 wherein said core additionally comprises super absorbent particles or fibers.

44. The article of claim 30 wherein said core additionally comprises super absorbent particles or fibers.

45. The article of claim 31 wherein said core additionally comprises super absorbent particles or fibers.

46. The article of claim 29 wherein said apertured film is three dimensional.

47. The article of claim 30 wherein said apertured film is three dimensional.

48. The article of claim 31 wherein said apertured film is three dimensional.

49. The article of claim 29 wherein said article is arranged to be worn by a male and wherein said article has front portion, a rear portion and a longitudinal central axis, said longitudinal central axis having a center located between said front portion and said rear portion, said fluid intake zone being located on said central axis in said front portion, said composite fluid acquisition system being located over said core and under said top sheet at said fluid intake zone.

50. The article of claim 30 wherein said article is arranged to be worn by a male and wherein said article has front portion, a rear portion and a longitudinal central axis, said longitudinal central axis having a center located between said front portion and said rear portion, said fluid intake zone being located on said central axis in said front portion, said composite fluid acquisition system being located over said core and under said top sheet at said fluid intake zone.

51. The article of claim 31 wherein said article is arranged to be worn by a male and wherein said article has front portion, a rear portion and a longitudinal central axis, said longitudinal central axis having a center located between said front portion and said rear portion, said fluid intake zone being located on said central axis in said front portion, said composite fluid acquisition system being located over said core and under said top sheet at said fluid intake zone.

52. The article of claim 29 wherein said article is arranged to be worn by a female and wherein said article has front portion, a rear portion and a longitudinal central axis, said longitudinal central axis having a center located between said front portion and said rear portion, said fluid intake zone being located on said central axis at approximately said center, said composite fluid acquisition system being over said core and under said top sheet at said fluid intake zone.

53. The article of claim 30 wherein said article is arranged to be worn by a female and wherein said article has front portion, a rear portion and a longitudinal central axis, said longitudinal central axis having a center located between said front portion and said rear portion, said fluid intake zone being located on said central axis at approximately said center, said composite fluid acquisition system being over said core and under said top sheet at said fluid intake zone.

54. The article of claim 31 wherein said article is arranged to be worn by a female and wherein said article has front portion, a rear portion and a longitudinal central axis, said longitudinal central axis having a center located between said front portion and said rear portion, said fluid intake zone being located on said central axis at approximately said center, said composite fluid acquisition system being over said core and under said top sheet at said fluid intake zone.
